(19) United States
(12) Patent Application Publication
(10) Pub. No.: US 2004/0122381 A1
(43) Pub. Date: Jun. 24, 2004

(54) Medical flushing and suctioning device

(75) Inventor: Steffen Arnold, Blundenz (AT)

Correspondence Address: CHRISTINA HILDEBRAND, NORRIS, MCLAUGHLIN & MARCUS, 220 EAST 42ND STREET - 30TH FLOOR, NEW YORK, NY, 10017, US

(73) Assignee: A.M.I. (Agency for Medical Innovations GmbH), Gotzis (AT)

(21) Appl. No.: 10/699,970

(22) Filed: Nov. 3, 2003

(30) Foreign Application Priority Data
Nov. 6, 2002 (DE) 102 51 598.0

(51) Int. Cl. A61M 35/00

(52) U.S. Cl. 604/290

(57) ABSTRACT

A medical flushing and suctioning device for flushing and suctioning off body areas in an operating area is described, which consists of a flushing and suctioning lancet and a connected valve arrangement with switchable valves, which depending on the valve position supply a flushing fluid to the flushing and suctioning lancet or switch the lancet into a suctioning function. For improved handling of the device, the invention provides that all functional parts are combined in a device that can be manually held.

### RELATED APPLICATIONS

The benefit of priority of the German patent application number 102 51 598.0 filed on Nov. 6, 2002 in the name of the inventor, is hereby claimed.

### BACKGROUND OF THE INVENTION

1. Field of the Invention

The invention relates to a medical flushing and suctioning device.

2. Description of the Related Art

Medical flushing and suctioning devices are used for flushing wound areas, whereby these wound areas can be located in exterior regions as well as in interior regions of the body. If wound areas are to be flushed and suctioned off with surgical tools in the interior space of the body, then an endoscopic flushing-suction tube is attached to the medical flushing and suctioning device.

A flushing and suctioning device of the aforedescribed type designed for onetime use is known in the art.

A valve arrangement connected to two different hoses is connected to a lancet which can be used both as a flushing channel and as a suctioning channel. A flushing fluid can be introduced into the body opening through a corresponding flushing hose by actuating one valve. After that valve is closed, the other valve can be opened and the fluid previously introduced into the body opening is suctioned off.

One disadvantage of this arrangement is that the lancet is used both for flushing and for suctioning, which can lead to contamination of the surgical area. If the area is suctioned off first and thereafter flushed, then the suctioned-off particles are initially reintroduced into the wound area during the first flushing process and then suctioned off again.

Another disadvantage of this arrangement is that the valve arrangement cannot be reliably operated. When remotely controlled, it may not be preventable that both the flushing valve and the suction routes are actuated simultaneously, in which case the flushing flow in the valve body is immediately suctioned off. The body area is then not flushed.

Another disadvantage of the conventional medical flushing and suctioning device is that a relatively complex apparatus has to be operated. A double-acting pump is located in a separate stationery housing. The pump can perform the flushing and suctioning operation with a large flow volume. This requires a dedicated machine which requires a separate location in the operating room. This type of medical flushing and suctioning device has been successfully employed for flushing and suctioning off large fluid volumes. However, if smaller-sized wound areas or only small fluid volumes have to be flushed, then the complex operation of the conventional arrangement cannot be justified. This arrangement is designed for onetime use, whereby after the use the entire valve unit together with the suctioning lancet and all feed hoses have to be replaced.

### SUMMARY OF THE INVENTION

It is therefore an object of the invention to improve a medical flushing and suctioning device of the aforedescribed type so that an improved effect can be achieved with a significantly less complex machine which can also be manufactured more cost-effectively.

The solution of the object of the invention is characterized in that the at least one flushing container and a separate therefrom a collection container is arranged on a manually operated suction and flushing pump.

With the disclosed technical teachings, an essential advantage is achieved in that the flushing and suctioning device combines all functional parts in a single handheld device. The suctioning and flushing pump of the invention contains the flushing pump required for generating a flow of flushing fluid, and simultaneously also separate therefrom a suction pump required for suctioning from the wound area.

Each pump has its own container. This provides the advantage that the flushing fluid is drawn in from the flushing container by the flushing pump and transported through the flushing-suction tube to the wound area, and that after switching the manual actuator on the suction-pressure pump, the fluid that was previously flushed into the wound area is suctioned off again by using an additional manually operated suction pump and transferred to the collection container arranged on the suction-pressure pump.

According to a preferred embodiments of the invention, at least one of the collection containers is formed so as to be removable and hence exchangeable.

According to another embodiment of the invention, the flushing container may also be removably and exchangeably arranged on the suctioning and pressure pump. However, this is not to be understood as a limitation of the invention. The flushing container may also be directly integrated in the suction-pressure pump so as to form a single unit therewith.

According to the invention, the suctioning and pressure pump is also designed for onetime use, i.e., it is disposed of after onetime use.

Making the collection container removable has the advantage that the tissue fragments suctioned off from the wound area and the germ-contaminated fluids and the like can be easily analyzed by closing off the collection container after unscrewing it from the suction-pressure pump and examining the contents in a suitable laboratory.

In a preferred embodiments of the invention, one dedicated suction pump is provided for suctioning, and another dedicated pump for flushing. The two pumps are operated alternatingly, more particularly by a slider that is arranged on the manually operated lever of the suction-pressure pump.

Depending on the slider position on this lever, either the flushing or the suction pump is operated, but not both.

However, the invention is not limited to the arrangement with two separate pumps. According to another embodiment of the invention, the flushing pump is eliminated and the flushing container is placed under an overpressure, whereby the fluid is introduced into the surgical area solely by operating a manual valve.

According to another preferred embodiment of the invention, the flushing-suction tube can have two mutually separated channels. The larger-sized channel is suitable for suctioning off the larger wound and tissue particles, whereas the other smaller-sized channel is provided for supplying the flushing fluid to the surgical area. This has the advantage that no contaminating germs are introduced into the flushing circulation, as is the case with conventional devices.

The subject matter of the present invention is not limited to subject matter recited in the individual claims, but can also be derived from the combination of the individual claims.

All description and features disclosed in the application documents, including the abstract, in particular the three-dimensional design depicted in the drawings, are claimed as part of the invention, as far as they are novel over the prior art, either separately or in combination.

The invention will be described hereinafter with reference to the drawings which depict only a single embodiment. Additional features and advantages of the invention can be deduced from the drawings and their description.

Other aspects of the present invention will become apparent from the following detailed description considered in conjunction with the accompanying drawings. It is to be understood, however, that the drawings are intended solely for purposes of illustration and not as a definition of the limits of the invention, for which reference should be made to the appended claims.

### BRIEF DESCRIPTION OF THE DRAWINGS

It is shown in:

FIG. 1 a cross-sectional view of a suction-pressure pump according to the invention;

FIG. 2 a partially disassembled suction-pressure pump according to FIG. 1;

FIG. 3 a cross-section through the suction pump;

FIG. 4 a front view of the suction pump of FIG. 3;

FIG. 5 a cross-section through the flushing pump;

FIG. 6 a front view of the flushing pump of FIG. 5;

FIG. 7 a cross-section through the lever;

FIG. 8 a top view of the lever according to FIG. 7;

FIG. 9 a side view of the flushing-suction tube;

FIG. 10 a front view of the flushing-suction tube of FIG. 9.

### DETAILED DESCRIPTION OF THE PRESENTLY PREFERRED EMBODIMENTS

The Suction and Pressure Pump 1

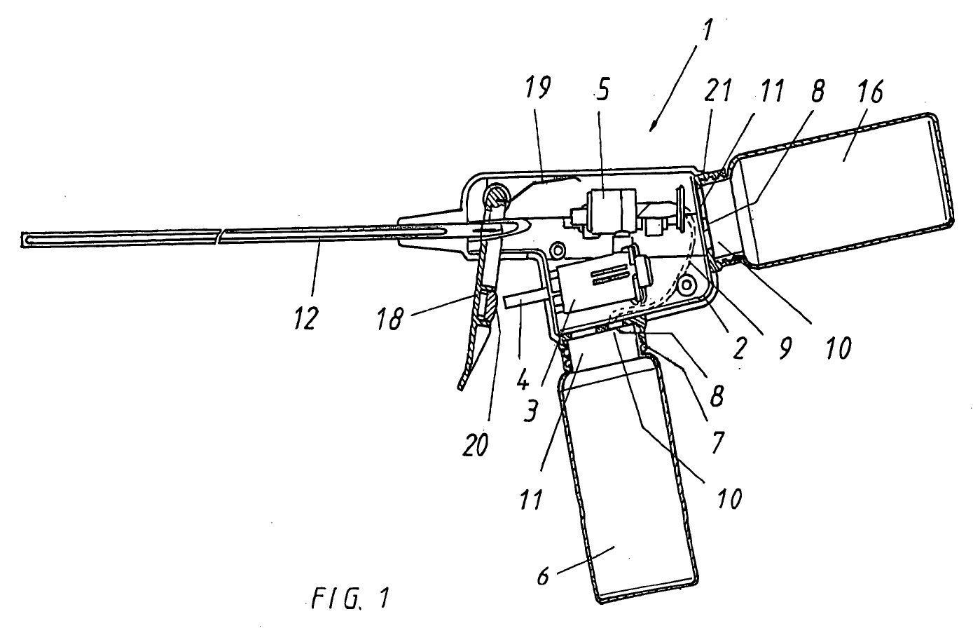

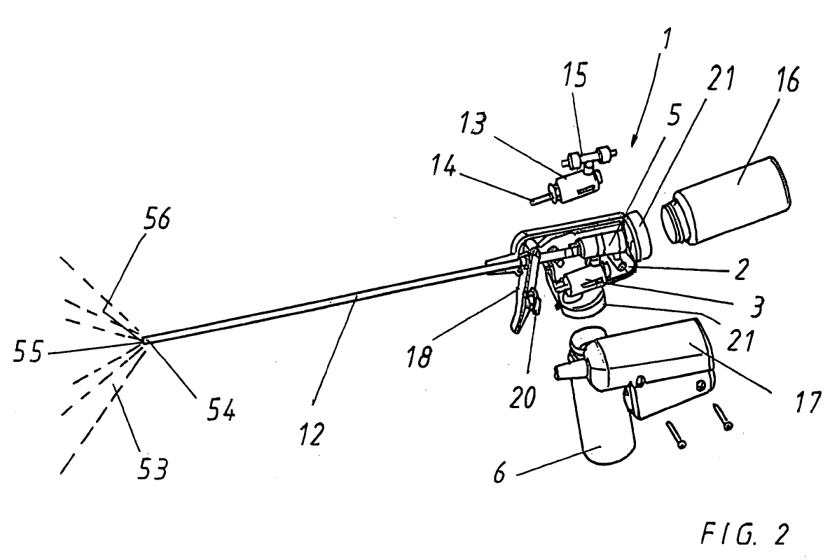

As shown in FIGS. 1 and 2, the suction-pressure pump 1 according to the invention consists essentially of a housing 2 with two housing shells that can be screwed together, wherein only the front housing shell 17 is depicted in FIG. 2.

Two independently actuatable pumps 3, 13 are arranged in the interior of the housing, whereby one pump is implemented as a suction pump 3 and the other pump as a flushing pump 13.

A shoulder 21 with a thread 7 is arranged on the bottom side of the housing 2. A collection container 6 which has an upper seal 8 and an opening 10 for insertion of the hose 9 (not shown) is screwed onto this thread. Also provided is a ventilation valve 11 which ensures that the fluid collected in the collection container 6 does not inadvertently leak out when the suction-pressure pump is placed on its side.

The Suction Pump 3

The valve block 5 is associated with the suction pump 3; the pump is actuated via a plunger 4 which is activated by a lever 18 which is rotatably supported with the spring 19. The suction pump 3 consists essentially of a cylinder 28 in which a piston 29 which is spring-biased by spring 30 is displaceably positioned. The piston 29 is connected with a plunger 4 which is activated by the aforedescribed lever 18.

This represents a single-acting pump because the cylinder chamber is connected via the fitting 26 with the valve block 5. The valve block 5 consists of two independently operating valve chambers 22, 23.

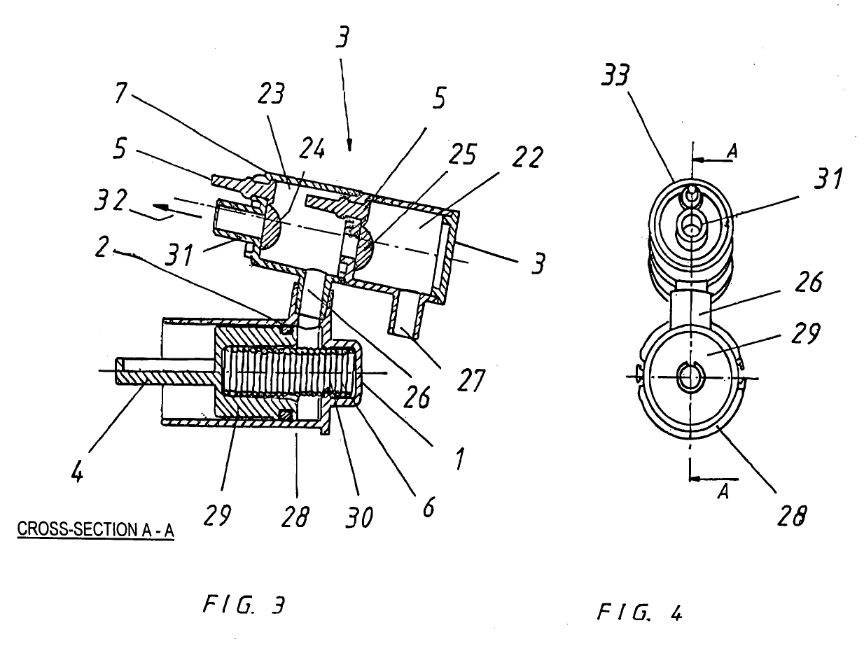

If the plunger 4 is relieved in the position depicted in FIG. 3, then the piston 29 moves to the left and draws fluid into the cylindrical chamber 1 through the fitting 26. The valve 24 disposed in the valve chamber 23 opens as a result and draws fluid into the valve chamber 23 in a direction opposite to the depicted direction of arrow 32. The valve 25 is simultaneously closed. After the valve chamber 23 is filled by the suction stroke, the valve 25 is opened when the plunger 4 is actuated next in the opposite direction, and the valve chamber 22 is filled. The suctioned-off material flows from the valve chamber 22 through the fitting 27 and the hose 9 into the collection container 6.

The illustrated valves 24, 25 are implemented as valve flaps made out of silicone with a large cross-sectional area.

The Flushing Pump 13

The flushing pump 13 is located in the rear section of the housing and is shown separately in FIG. 2 for sake of clarity. The pump 13 also has a plunger 14, and a valve block 15 is disposed at the top of the cylinder, with the outlet of the valve block connected through a hose (not shown) to the opening 10 of the flushing container 16.

This flushing container also has, in the same manner as described above, a seal 8 with a ventilation valve 11 and is screwed with a thread onto the shoulder of the housing of the suction-pressure pump 1.

By separately arranging a suction pump 3 with a flushing pump 13, the suction pump 3 can advantageously have a large suction cross-section, so that large volumes can be suctioned off and coarse particles can be entrained. Conversely, the flushing pump 13 requires only a relatively small flushing cross-section, transporting correspondingly less fluid into the wound area. Accordingly, only one pump has to be sized with a large cross-section.

For this reason, the valves in valve block 15 of the flushing from 13 also have a smaller size.

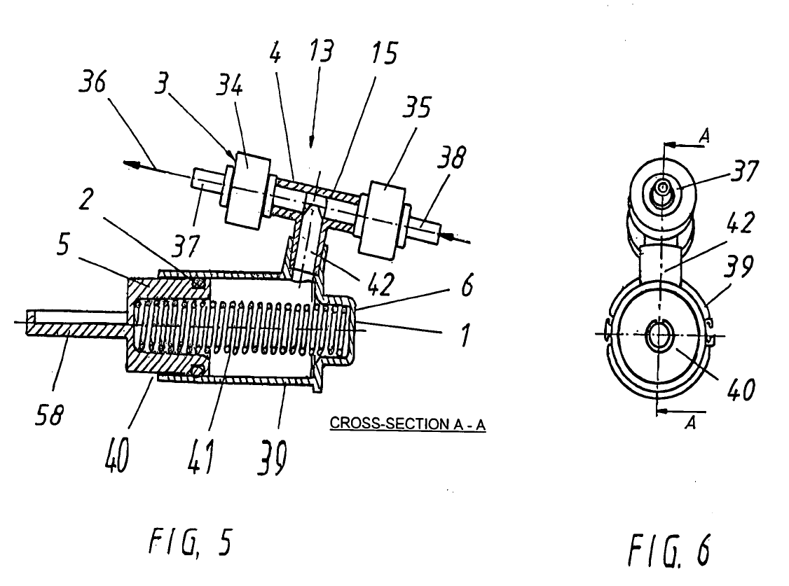

Accordingly, the flushing pump 13 depicted in FIGS. 5 and 6 includes again a cylinder 39, in which the piston 40 which is spring-biased by spring 41 moves back and forth. The front end of the piston again includes a plunger 58.

The cylinder chamber is connected via fitting 42 with the valve block 15 which again consists of two valve chambers 34, 35. The functionality is identical to that described with reference to the suction pump 3 and the FIGS. 3 and 4.

When a corresponding compression stroke opposing the force of the spring 48 is applied to the plunger 58, the volume of the cylinder chamber is reduced and the flushing volume contained in the cylinder chamber is sprayed out of the fitting 37 in the direction of arrow 36, whereby the valve in the valve chamber 35 is closed and the valve in the valve chamber 34 is opened.

In the next suction stroke, the valve in the valve chamber 34 is closed and the valve in the valve chamber 35 is opened, drawing fluid through the fitting 38 from the flushing container 16 in the direction of the arrow.

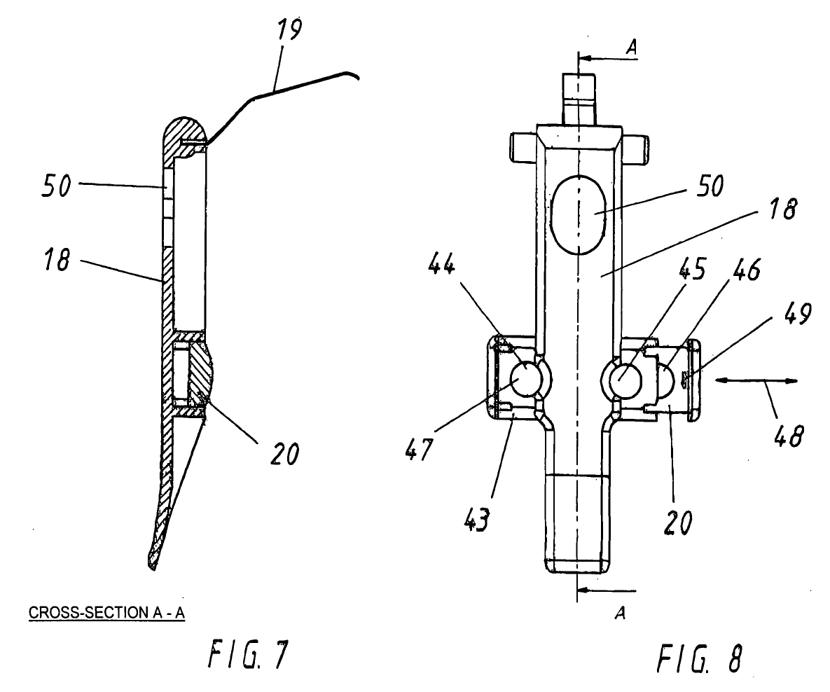

FIGS. 7 and 8 show the spring-biased lever 18 which guarantees that only a single pump, i.e., either the suction pump 3 or the flushing pump 13 can be actuated. This is achieved by fixedly arranging on the lever 18 a transverse member 43 with two spaced-apart bores 44, 45.

A movable slider 20 which also has two bores 46, 47 matching the bores 44, 45 is disposed in this transverse member 43.

In the displacement position depicted in FIG. 8, the bore 47 in the slider is aligned with the bore 44 in the transverse member of the lever 18, so that this bore forms an unobstructed passageway. For example, the plunger 4 of the suction pump 3 engages in this bore, so that the suction pump cannot be actuated when the lever 18 is moved manually.

Likewise, as a result of the offset of the two bores 45, 46, the bore 45 in the transverse member is closed off by the slider 20, and the plunger 58 of the flushing pump 13 contacts the closed section of bore 45 and is therefore actuated.

However, if the slider 20 is moved to the left in the direction of arrow 48, then the bore 44 is closed and the bore 45 is opened. In this displacement position, the suction pump 3 is placed in an operational state.

The arrangement of slider 20 makes it therefore impossible to actuate the two pumps simultaneously, as is possible with conventional devices.

Moreover, a snap-in projection 49 is disposed in the slider 20 for locking the two displacement positions.

Moreover, the lever 18 has a larger opening 50 through which the flushing and suction tube 12 extends, which will be described hereinafter in more detail.

The flushing and suction tube 12

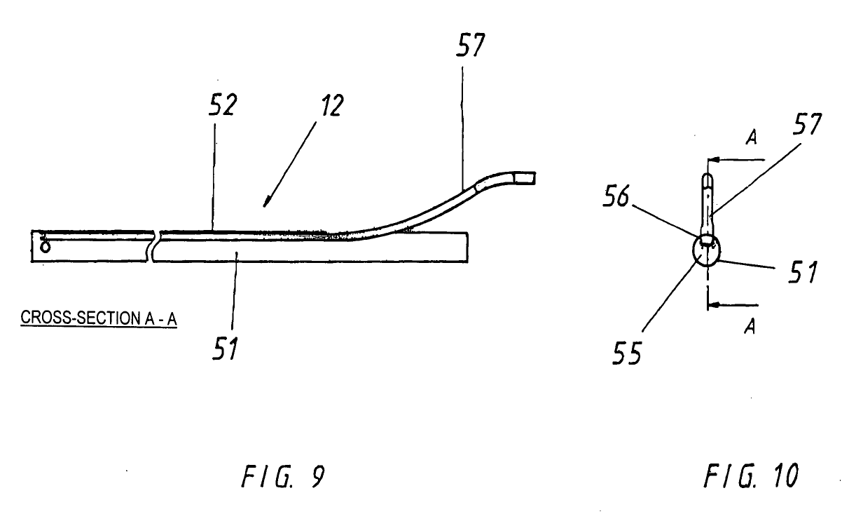

According to a preferred embodiment depicted in FIGS. 9 and 10, the flushing-suction tube 12 is formed in two parts and consists of a larger-sized suction tube 51 and a flushing tube 52 which is routed in parallel and connected to the suction tube 51. The two parts 51, 52 are connected with each other, whereby different possibilities exist:

In a first embodiment, the flushing tube 52 can be guided in the interior space of the suction tube 51, so that—according to FIG. 10—the flushing nozzle 56 of the flushing tube 52 is located inside the suction bore 55 of the suction tube 12.

According to another embodiment of the invention, the flushing tube 52 can also be guided outside the suction tube 51, so that the flushing nozzle 56 is also located outside the suction bore 55 of the suction tube 12.

FIG. 2 illustrates the superior flushing effect achieved with the flushing pump 13. The flushing nozzle 56 creates a flushing fan 53 which can be spread over a large section of the surgical area. Large tissue particles and other particles are suctioned off through the large-size front suction bore 55. However, if the suction bore 55 is blocked, then lateral relief bores 54 are arranged on the front side of the flushing-suction tube 12 so as to still enable a suction process.

A large area flushing fan 53 for optimally flushing the wound area to be cleaned can be generated by guiding the flushing nozzle 56 in the front region of the suction bore 55 and also beveling the flushing nozzle 56.

The suction-pressure pump of the invention can advantageously be manufactured using relatively inexpensive parts which are suitable for onetime use, which makes the apparatus for this application relatively simple.

No separately installed pumps and corresponding suction and flushing containers are required, since all components for flushing and suctioning off wound areas are combined in a single device capable of being manually operated.

Thus, while there have been shown and described and pointed out fundamental novel features of the invention as applied to a preferred embodiment thereof, it will be understood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. Substitutions of elements from one described embodiment to another are also fully intended and contemplated. It is also to be understood that the drawings are not necessarily drawn to scale but that they are merely conceptual in nature. It is the intention, therefore, to be limited only as indicated by the scope of the claims appended hereto.

What is claimed is:

1. A medical flushing and suctioning device for flushing and suctioning off a wound area, comprising
a flushing and suctioning lancet;
a connected valve arrangement having switchable valves to provide several valve positions;
wherein, depending on one valve position, a flushing fluid is provided to the flushing and suctioning lancet and the lancet is switched to a suction function;
and wherein the flushing and suctioning device is an integral device including all functional parts, capable of being manually held.

2. The medical flushing and suctioning device according to claim 1, further comprising at least one flushing container and a separate collection container arranged on a manually operated suction pump and flushing pump.

3. The medical flushing and suctioning device according to claim 1, wherein the device comprises a flushing pump for generating a flow of flushing fluid, and, a suction pump suctioning off wound areas.

4. The medical flushing and suctioning device according to claim 3, wherein the flushing pump and the suction pump are each associated with a container.

5. The medical flushing and suctioning device according to claim 2, wherein one of the at least one collection container is removable and sealable.

6. The medical flushing and suctioning device according to claim 2, wherein the flushing container is removable and interchangeably arranged on the suction and flushing pump.

7. The medical flushing and suctioning device according to claim 2, wherein the flushing container is non-removably integrated in the device.

8. The medical flushing and suctioning device according to claim 2, wherein both suction and flushing pumps are operated alternatingly.

9. The medical flushing and suctioning device according to claim 8, wherein the alternating operation of the suction pump and flushing pump is performed with a slider which is arranged on a manually operated lever on the device.

10. The medical flushing and suctioning device according to claim 1, further comprising a flushing-suction tube including a flushing channel and a suction channel.

11. The medical flushing and suctioning device according to claim 8, further comprising a spring-biased lever operatively connected to the suction pump and the flushing pump, the spring-biased lever is rotatable to one side and arranged in a housing, the lever being connected with a transverse member having two spaced-apart bores which can optionally be brought into an overlapping or covering disposition with corresponding bores by a slider which is moveable in the transverse member, whereby plungers associated to the suction pump and the flushing pump are each aligned with the bores in the transverse member.
